(19) United States
(12) Patent Application Publication
(10) Pub. No.: US 2009/0234281 A1
(43) Pub. Date: Sep. 17, 2009

(54) Fluid feeder and balloon catheter

(75) Inventor: Yutaka YANUMA, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/046,744

(22) Filed: Mar. 12, 2008

(51) Int. Cl. A61M 29/02

(52) U.S. Cl. 604/97.02

(57) ABSTRACT

A fluid feeder which feeds a fluid into a balloon made of an elastic material so as to inflate, the fluid feeder provided with: a cylinder that is formed in a cylindrical shape provided with a first port and a second port on each end thereof and contains the fluid; a plunger that is inserted into the cylinder via the second port in a freely advancing and retracting manner in an axial direction, in order to push out the fluid contained in the cylinder from the first port to the outside of the cylinder; an adjustor that is attached to the plunger and regulates a moving distance of the plunger so as to inflate the balloon to a predetermined diameter; and a fixing portion that is provided on the cylinder and fixes the plunger onto the cylinder to a position which corresponds to the moving distance regulated by the adjustor, wherein:
the adjustor has a plurality of engaging members which regulates the moving distance corresponding to a plurality of the different inflated diameters, and the fixing portion has an engaged portion which engages with the engaging members to fix the plunger.

### BACKGROUND OF THE INVENTION

1. Field of the Invention

The present invention relates to a fluid feeder and a balloon catheter which are employed by feeding fluid into a balloon.

2. Description of Related Art

In general, various types of balloon catheters provided with a balloon in the vicinity of a distal end of the catheters are employed in a medical field, for example, urological catheters provided with a balloon for medical use such as indwelled urinary catheters, endotracheal tubes, digestive tract catheters and cardiac balloon catheters for cardiac pumping.

When the balloon of the balloon catheters is inflated, in general, a fluid feeder (such as a syringe of medical use) is fitted onto a port located at a handheld side which is connected to the balloon so as to flow a fluid. The balloon is inflated to a required volume by injecting the fluid.

When a gallstone is removed with the balloon catheters described above, a balloon 100 is inflated with a slightly larger diameter than that of a bile duct 110. Thereafter, a gallstone 111 is carried closer to the entry of the bile duct by scraping the gallstone 111 with the balloon 100.

Due to the narrow diameter of the entry of the bile duct, the balloon catheters carrying the gallstone can not be withdrawn from the bile duct without reducing the diameter of the balloon 100. Therefore, in normal practice, an assistant operates the fluid feeder in conjunction with the balloon 100 being pulled by a user; hence the pulling operation is performed by deflating the balloon 100.

However, it is not easy to synchronize the operations of the user and the assistant. If the synchronized operation is not carried out correctly; in this case, if the deflation of the balloon 100 is too fast, the gallstones 111 is separated from the balloon 100 and left in the bile duct.

On the other hand, if the deflation of the balloon 100 is too slow, the balloon 100 may compress the exit of the bile duct 110, or the balloon 100 may rupture.

Further, as the diameter of the balloon becomes smaller and smaller, the diameter will change significantly with a slight operation of the fluid feeder. This is problematic as adjustment of the diameter of the balloon to a desirable size becomes difficult as a result.

### SUMMARY OF THE INVENTION

The present invention was conceived in view of the above-described circumstances, and has as its objective the provision of a fluid feeder which can adjust/control the diameter of a balloon to a desirable diameter regardless of the diameter of the balloon.

Another object of the present invention is the provision of a balloon catheter which can reliably adjust the diameter of the balloon to a desirable diameter.

According to a first aspect of the present invention, a fluid feeder which feeds a fluid into a balloon made of an elastic material so as to inflate, the fluid feeder provided with: a cylinder that is formed in a cylindrical shape provided with a first port and a second port on each end thereof, and contains the fluid; a plunger that is inserted into the cylinder via the second port in a freely advancing and retracting manner in an axial direction, in order to push out the fluid contained in the cylinder from the first port to the outside of the cylinder; an adjustor that is attached to the plunger and regulates a moving distance of the plunger so as to inflate the balloon to a predetermined diameter; and a fixing portion that is provided on the cylinder, and fixes the plunger onto the cylinder to a position which corresponds to the moving distance regulated by the adjustor; wherein:

the adjustor has a plurality of engaging members which regulates the moving distance corresponding to a plurality of the different inflated diameters, and the fixing portion has an engaged portion which engages with the engaging members to fix the plunger.

According to a second aspect of the present invention, a balloon catheter provided with a balloon made of an elastic material, and includes the fluid feeder of the present invention.

### BRIEF DESCRIPTION OF THE DRAWINGS

FIG. 1 shows the balloon catheter provided with the fluid feeder according to a first embodiment of the present invention.

FIG. 2 is a perspective view of the fluid feeder of the present invention.

FIG. 3 is a plain view of the fluid feeder of the present invention.

FIG. 4 is a cross-sectional view as seen from a line A-A of FIG. 3.

FIG. 5 shows an action of the plunger and the moving distance.

FIG. 6 is an example of a graph showing a relationship between the size of the diameter of the balloon and the moving distance of the plunger.

FIG. 7 shows the balloon catheter fixing onto an endoscope.

FIG. 8 shows the action of the balloon catheter 2 during use.

FIG. 9 shows the balloon in an inflated state.

FIG. 10 shows a state in which the adjustor and the engaged portion are engaged.

FIG. 11 shows an action of the engagement between the adjustor and the engaged portion to change to an ‘OFF’ state.

FIG. 12 shows an action of the engagement between the adjustor and the engaged portion to change to ‘OFF’ state.

FIG. 13 is an enlarged view of the adjustor and the engaged portion of a fluid feeder according to a second embodiment of the present invention.

FIG. 14 is an enlarged view of the adjustor and the engaged portion of a fluid feeder according to a modified examples of the present invention.

FIG. 15 is an enlarged view of the adjustor and the engaged portion of a fluid feeder according to a modified examples of the present invention.

FIG. 16A shows a state in which a gallstone is removed by a conventional balloon catheter.

FIG. 16B shows a state in which a gallstone is left in the vicinity an entry of a bile duct.

### DETAILED DESCRIPTION OF THE INVENTION

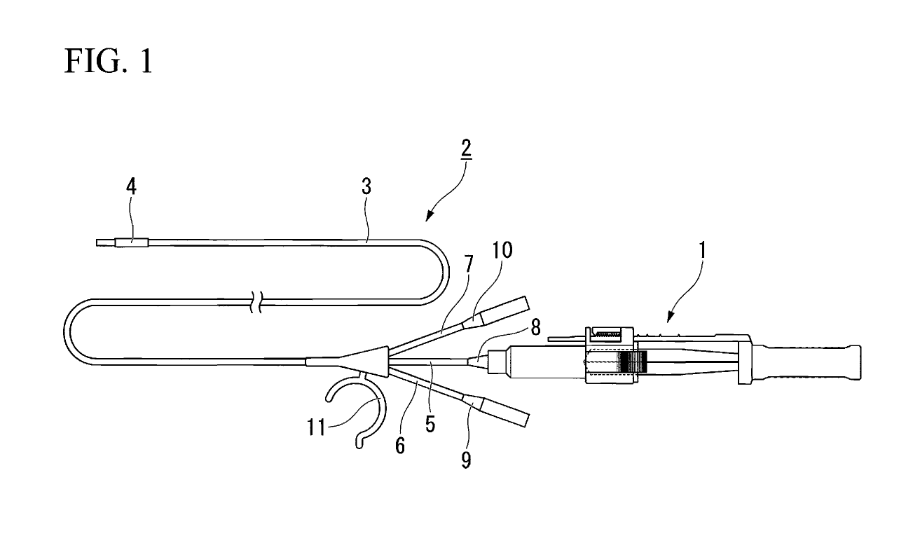

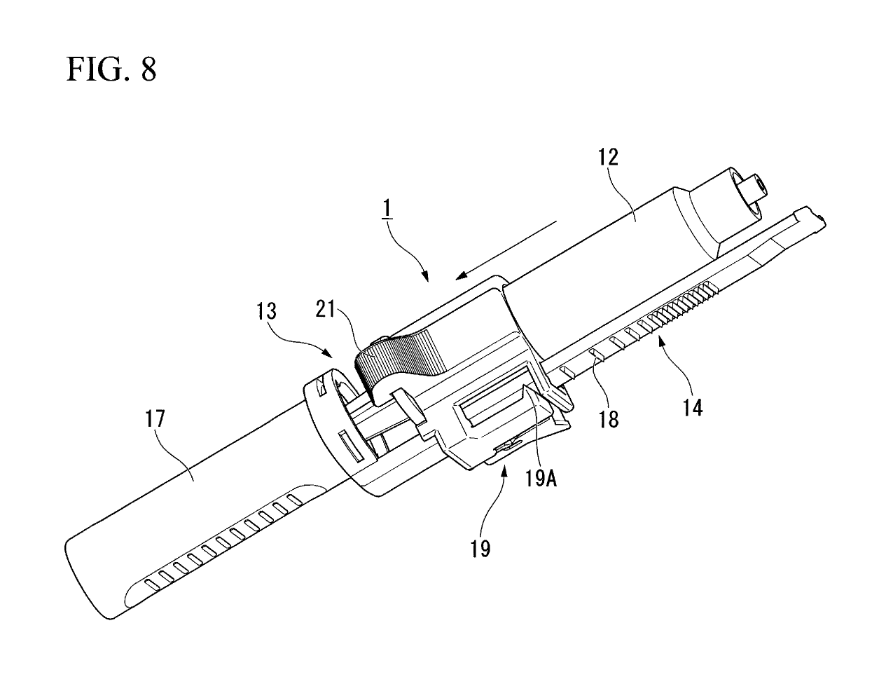

A fluid feeder according to a first embodiment of the present invention will be explained with reference to FIGS. 1 through 12.

FIG. 1 is a view showing a balloon catheter 2 provided with a fluid feeder 1 according to the present embodiment. The balloon catheter 2 includes a long flexible sheath 3, a balloon 4 provided in the vicinity of a distal end of the sheath 3, and the fluid feeder 1 provided at a proximal end of the sheath 3.

The sheath 3 is made of a flexible material, such as resin, and is provided with three lumens; a first lumen 5 for feeding fluid to a balloon 4, a second lumen 6 for inserting a guidewire which guides a distal end of the balloon catheter 2 to a desirable position of a body cavity of a patient, and a third lumen 7 for transporting various fluid, such as a contrast agent, into the body cavity of the patient.

A distal end of the first lumen 5 passes through an outer periphery surface of the sheath 3, and opens into the balloon 4. Distal ends of the second lumen 6 and the third lumen 7 open to the distal end of the sheath 3. However, the distal ends of the second lumen 6 and the third lumen 7 may also open at any position other than the distal end of the sheath 3.

The lumens 5, 6, 7 do not integrate each other, rather, they are provided within the sheath 3 independently. The three independent lumens extend and separate from the proximal ends, forming three ports: a first port 8, a second port 9 and a third port 10. The fluid feeder 1 is connected to the first port 8.

A proximal end of the sheath 3 also includes an anchor 11 which fixes the balloon catheter 2 at, for example, an endoscopic device or the like.

The balloon 4 is made of an elastic material so as to inflate by gradually expanding the diameter thereof when a fluid such as liquid or gas which is fed from the fluid feeder 1 accumulates inside thereof. As for the material made of the balloon 4, for example, natural rubber, synthetic rubber, polyurethane, polyamide elastomer, silicone, and the like can be employed suitably according to its purpose.

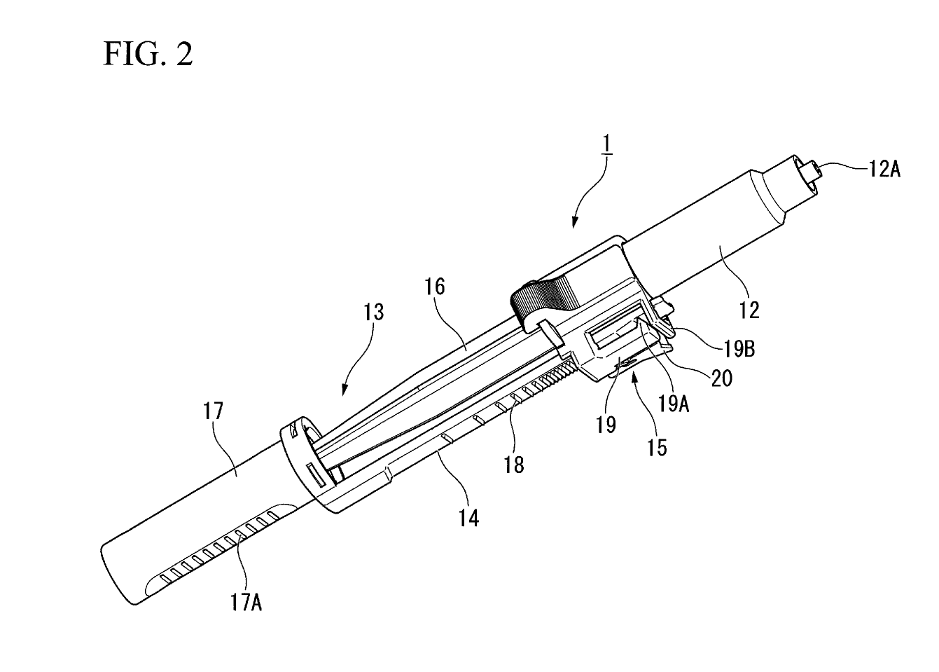

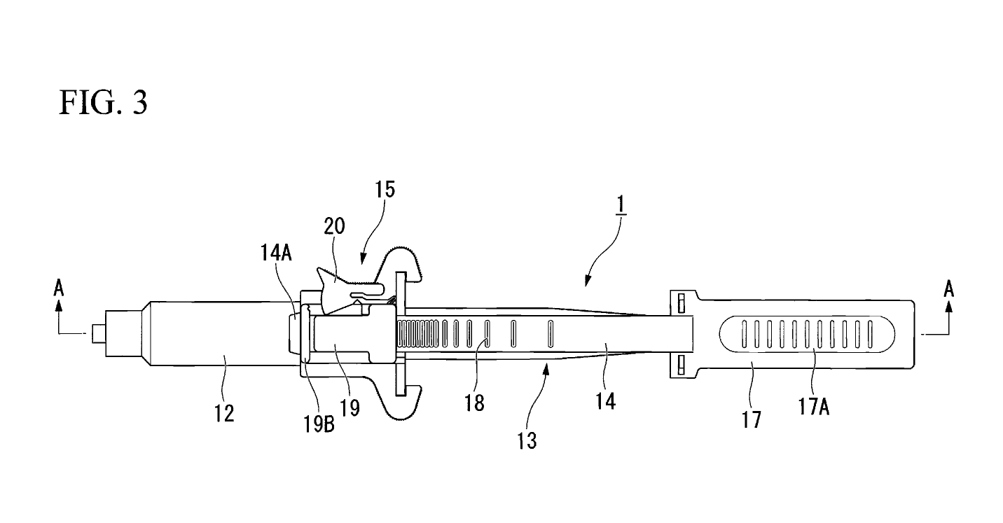

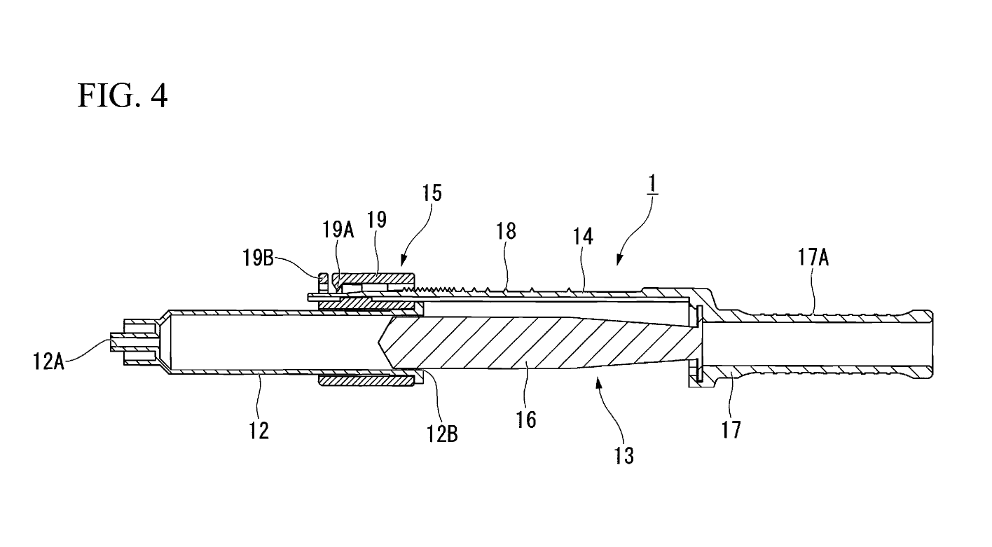

FIG. 2 is a perspective view of the fluid feeder 1, FIG. 3 is a plain view of the fluid feeder 1, and FIG. 4 is a cross-sectional view as seen from a line A-A of FIG. 3. As indicated in FIGS. 2 through 4, the fluid feeder 1 includes; a cylindrical-shaped cylinder 12, a plunger 13 which is inserted into the cylinder 12, an adjustor 14 provided on the plunger 13, and a fixing portion 15 provided on the cylinder 12.

The cylinder 12 is formed in a cylindrical shape, containing fluid inside a lumen thereof. The fluid contained may be a gas such as air, or a liquid, such as a saline solution. Both ends of the cylinder 12, a first end 12A at the distal end, and a second end 12B at the proximal end, are opened. The first end 12A engages with the first port 8 so that the shape of the first end 12A is constructed in a manner so as to allow engagement with the first port 8.

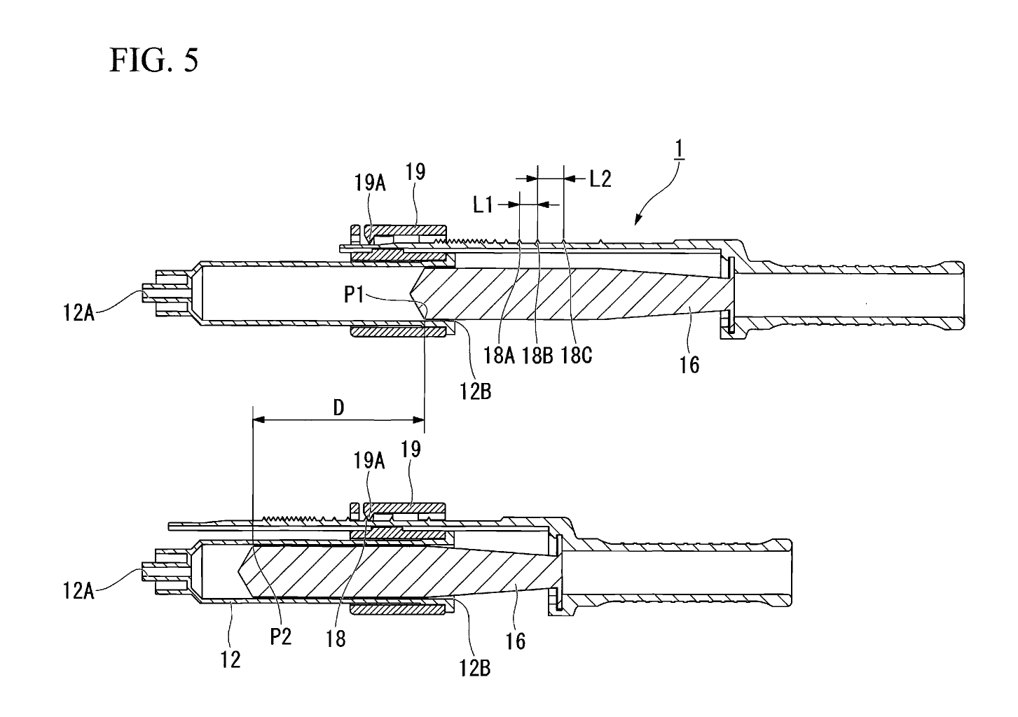

The plunger 13 includes a main body 16 which is inserted into the cylinder, and a grip 17 provided at a proximal side of the main body. A distal side of the main body 16 is inserted into the lumen of the cylinder 12, so as to advance and retract the main body 16 inside of the cylinder 12 along a longitudinal direction thereof. A shape of the distal end of the main body 16 is substantially the same as the inner diameter of the cylinder 12. As shown in FIG. 5, when the main body 16 is advanced toward the first end 12A of the cylinder 12, the fluid contained in the cylinder 12 is pushed out from the first end 12A.

A grip 17 is a member gripped by a user when the plunger 13 is operated, and there are no limits in the shape and material. The grip 17 in the proceeding embodiment is formed in a cylindrical-shape, provided with a slip resistance 17A for ease of gripping.

The adjustor 14 is a plate member extending from the grip 17 in a direction substantially parallel to the main body 16 of the plunger 13. A convex portion 14A is formed at a distal end of the adjustor 14 so as to prevent the plunger 13 from escaping from the cylinder 12.

A plurality of engaging projections 18 which regulate an moving distance of the plunger 13 are formed on a surface of the adjustor 14 opposite to a surface facing to the main body 16. A distance between each of the engaging projections 18 is set longer as the position of the projections 18 is closer to the proximal end of the plunger 13. For example, as shown in FIG. 5, a distance L2 between an engaging projection 18B and an engaging projection 18C located more closer to the proximal side of the plunger 13 with respect to the projection 18B is longer than that of L1 between an engaging projection 18A and the engaging projection 18B located closer to the proximal side of the plunger 13 with respect to the projection 18A.

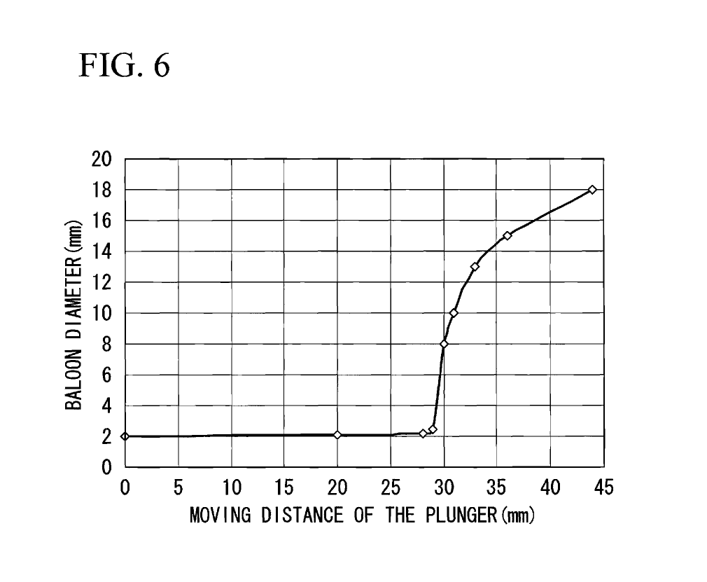

The distance between each of the engaging projections 18 is determined depending on the characteristics of the balloon 4 which are a target member for the fluid feeder 1 feeding a fluid thereinto. FIG. 6 shows an example of a graph indicating the relationship between the size of a diameter of the balloon and the moving distance of the plunger 13.

Note that ‘a moving distance’ refers to a distance D indicated by the same part of the plunger 13 in the fluid feeder 1 connected to the balloon catheter 2 in a state in which the balloon 4 is completely deflated. As shown in FIG. 5, the distance D is a distance between a reference position P1 which is a starting position of the plunger 13 and a position P2 after the movement of the plunger 13 completes.

It is preferable if the reference position P1 of the plunger 13 is set such that the distal end of the plunger 13 is positioned in the vicinity of the second end 12B of the cylinder 12. In this way, a wider range of the moving distance can be achieved. However, the present invention is not limited thereto; any positions may be set as the reference position P1. According to the present embodiment, the convex portion 14A of the adjustor 14 determines the position of the plunger 13 abutting a fall-off prevention portion of the fixing portion 15 as a reference position.

As shown in FIG. 6, the difference of the moving distance which is required by the plunger 13 for further inflating a pre-inflated balloon with a desirable diameter by a predetermined amount, for example by 1 mm, increases as the diameter of the pre-inflated balloon increases. Taking into consideration the relationship described above, if a distance (space) between the adjacent projections 18 is set so as to gradually increase as increasing a diameter of the balloon, it is possible to achieve an inflation of the diameter of the balloon which corresponds to each of the projections 18 with an equal interval, for example by 1 mm. Note that a position where the engaging projections 18 is formed may be altered depending on, for example, inflation and deflation characteristics of the balloon, an interval size for achieving a desirable diameter and a desirable size of diameter to be held after inflation, or the like.

The fixing portion 15 is disposed on an outer periphery of the cylinder 12, provided with an engaged portion 19 which engages with the engaging projections 18 of the adjustor 14, a switch 20 which changes the status of engagement between the engaged portion 19 and the adjustor 14 so as to engage and disengage, and a finger resting portion for a user to rest his/her fingers at the time of the operation.

The engaged portion 19 is positioned further outward in a radial direction of the cylinder 12 with respect to the engaging projections 18, and a projection 19A is protruded so as to oppose to the engaging projections 18. When the plunger 13 slides through the inside of the cylinder 12 and a proximal side face of the engaging projections 18 and a distal side face of the projection 19A comes into contact, a positional relationship between the plunger 13 and the cylinder 12 is maintained. At this time, the inflation of the balloon 4 is maintained with the inflated diameter corresponded to the moving distance of the plunger 13 which is regulated by the engaging projections 18.

As shown in FIGS. 2 and 3, a frame-shaped fall-off prevention portion 19B is disposed on the engaged portion 19, and the adjustor 14 is passed through the fall-off prevention portion 19B. When the plunger 13 is retracted toward the proximal end, the convex portion 14A of the adjustor 14 abuts onto the fall-off prevention portion 19B at a position where the distal end of the plunger 13 comes close to the second end 12B of the cylinder 12. As a result, the plunger 13 is no longer retracted further toward the proximal end so as to prevent the plunger 13 from escaping from the cylinder 12.

One end of the switch 20 is attached to the engaged portion 19 in a freely rotating manner about the end. When the switch 20 is rotated so as to interpose between the engaged portion 19 and the adjustor 14, the engaged portion 19 and the adjustor 14 are separated so as not to engage each other. This action will be described later.

The action of the balloon catheter 2 as designed above during use will not be explained.

First, an endoscope is inserted into a body cavity of a patient, and the distal end thereof is moved into the vicinity of a treatment target tissue.

At this time, the balloon catheter 2 should be ready for use. A user retracts the plunger 13 of the fluid feeder 1 toward the proximal side so as to position the plunger 13 at the reference point P1 described above, resulting a fluid flowing into the cylinder 12 to accumulate therein. Then the first end 12A of the cylinder 12 is connected to the first port 8.

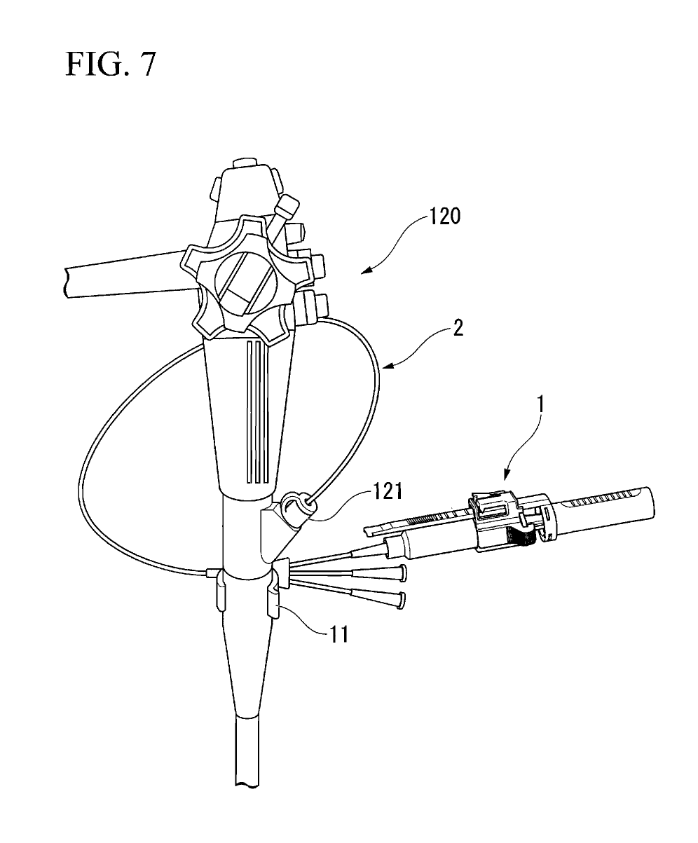

The user inserts the distal end of the balloon catheter 2 into an instrument channel of the endoscope (not shown) from a forceps port 121 of the endoscope 120 so as to protrude the distal end of the balloon catheter 2 from a distal end of the endoscope 120. Normally, an assistant operates the fluid feeder 1 by standing close to the user; however, the user may directly operate the fluid feeder 1 by himself/herself, by fixing a handheld side of the balloon catheter 2 to the endoscope 120 with the anchor 11, as shown in FIG. 7.

When the balloon 4 is inflated, the user grips the grip 17 of the plunger 13 resting his/her fingers on the finger resting portion 21. The plunger 13 is advanced into the syringe 12 by pulling the syringe 12 relative to the plunger 13. The projection 19A of the engaged portion 19 advances toward the engaging projections 18 positioned further to the proximal side, by subsequently clicking into the space between the adjacent engaging projections 18 of the adjustor 14. Simultaneously, a fluid contained in the cylinder 12 is pushed out by the plunger 13, feeding into the balloon 4 via the first port 8 and the first lumen 5 so as to inflate the balloon 4 as shown in FIG. 9.

When the user stops the advancing operation of the plunger 13, the balloon 4 is deflated so that a pressure which pushes the fluid back to the syringe 12 exerts onto the plunger 13. As a result, the engaging projections 18 of the adjustor 14 move toward the proximal end. Then, a proximal side slant face of the engaging projections 18 positioning closer to the distal end with respect to the projection 19A and closest to the projection 19A, comes into a contact with a distal side slant face of the projection 19A causing the plunger 13 to stop. Accordingly, the moving distance of the plunger 13 is maintained constant, so that the diameter of the balloon 4 is regulated and maintained as a set diameter size corresponding to the moving distance.

When the diameter of the balloon 4 is changed, the plunger 13 is advanced and retracted with respect to the cylinder 12 by the aforementioned action, so as to engage the projection 19A of the fixing portion 15 and the engaging projections 18 of a desirable position. Hence, the moving distance of the plunger 13 is regulated by the set position of the corresponding engaging projections, and the diameter of the balloon 4 changes according to the moving distance. When the plunger 13 advances and retracts and the engaged portion 19 rides over the engaging projections 18, the operator will feel a click. Hence, a user can easily recognise the number of levels shifted (in other words, a number of the engaging projections 18 in which the projection 19A has ridden over) without actually seeing a manipulation of the device. Accordingly, a diameter of the balloon 4 can be easily regulated. Further, if the plunger 13 is quickly retracted and the projection 19A of the fixing portion 15 is engaged to the engaging projections 18 at the position where the retraction of the plunger 13 is completed, in order to create a negative pressure state inside of the cylinder 12. As a result, the balloon 4 can be deflated faster.

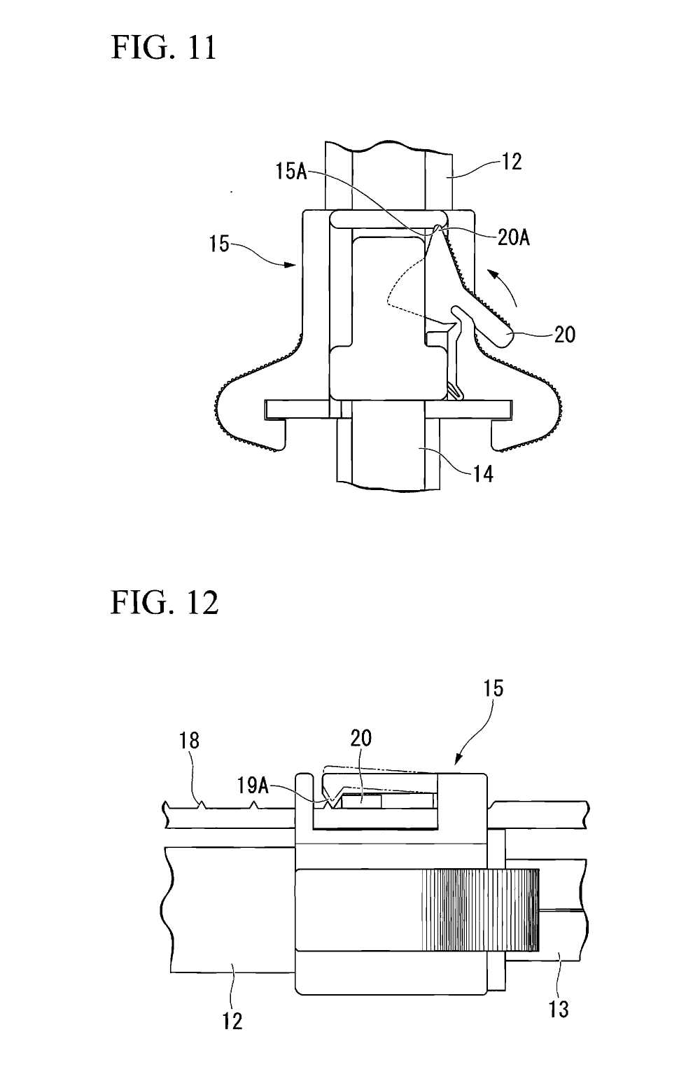

When the balloon 4 is completely deflated, the switch 20 is pushed in so as to insert between the projection 19A and the engaging projections 18, as shown in FIGS. 11 and 12. Then as shown in FIG. 12, the projection 19A is pushed up so as to separate the projection 19A from the engaging projections 18 by the switch 20. Accordingly, the projection 19A and the engaging projections 18 are no longer engaged, changing the status of the fixing portion 15 to ‘OFF’. As shown in FIG. 11, the ‘OFF’ state can be maintained by engaging an end portion 20A of the switch 20 onto the convex portion 15A provided on the fixing portion 15. At this ‘OFF’ state, the plunger 13 can be smoothly slide within the cylinder 12.

When the fixing portion 15 enters the ‘OFF’ state, the plunger 13 is pushed back by a deflation of the balloon 4 so that the balloon 4 completely deflates without the user operating the plunger 13. Alternatively, the ‘OFF’ state may be employed when the plunger 13 is pulled toward the proximal end at the aforementioned preparation stage of the balloon catheter 2 prior to use.

According to the fluid feeder 1 of the present embodiment, the moving distance of the plunger 13 is regulated by the engaging projections 18 provided on the adjustor 14 so as to inflate the diameter of the balloon 4 to a size which corresponded to the moving distance. Then, by engaging the engaging projections 18 and the projection 19A of the fixing portion 15, the inflated diameter of the balloon 4 is maintained. By virtue of the engaging action, the diameter of the balloon can be accurately regulated, even within a relatively small range of a diameter such that a diameter changes significantly with slight movement of the plunger.

Furthermore, since a distance between adjacent engaging projections is set to be increased as the moving distance of the adjustor 14 increases, it is possible to set the change of diameter (the difference by which the diameter changes) of the balloon 4 to be the same. The change of diameter is caused by clicking the engaging projections 18 which engages the projection 19A of the engaged portion 19 into the space between the next adjacent space toward the distal or proximal ends. Accordingly, the inflating diameter of the balloon 4 can be easily regulated at an equal interval, for example by 1 mm.

Furthermore, since the inflated state with a desirable diameter of the balloon 4 is maintained by the fixing portion 15, it is not necessary to provide a mechanism such as a faucet and the like, between the fluid feeder and the first port 8 for preventing a back flow of a fluid into the cylinder 12. Therefore, the structure of the balloon catheter 2 can be simplified, achieving lowering a manufacturing cost by employing the fluid feeder 1.

The present embodiment described examples in which the balloon catheter 2 was inserted into the endoscope 120. However, the present invention is not limited thereto; the balloon catheter 2 may be used without being fixed/inserted into the endoscope if a treatment is performed without the endoscope.

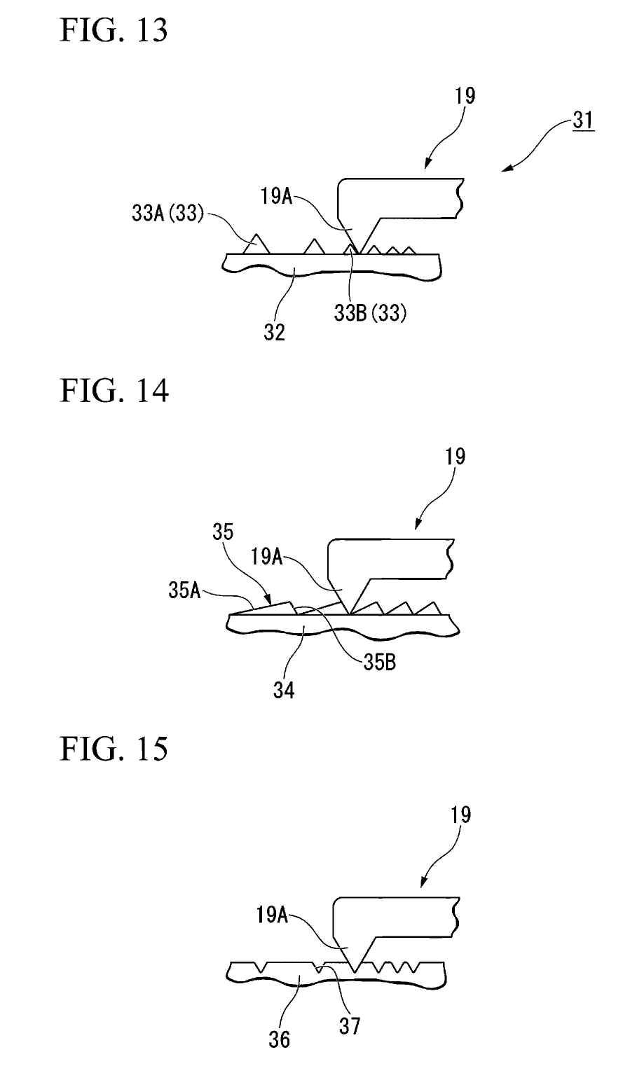

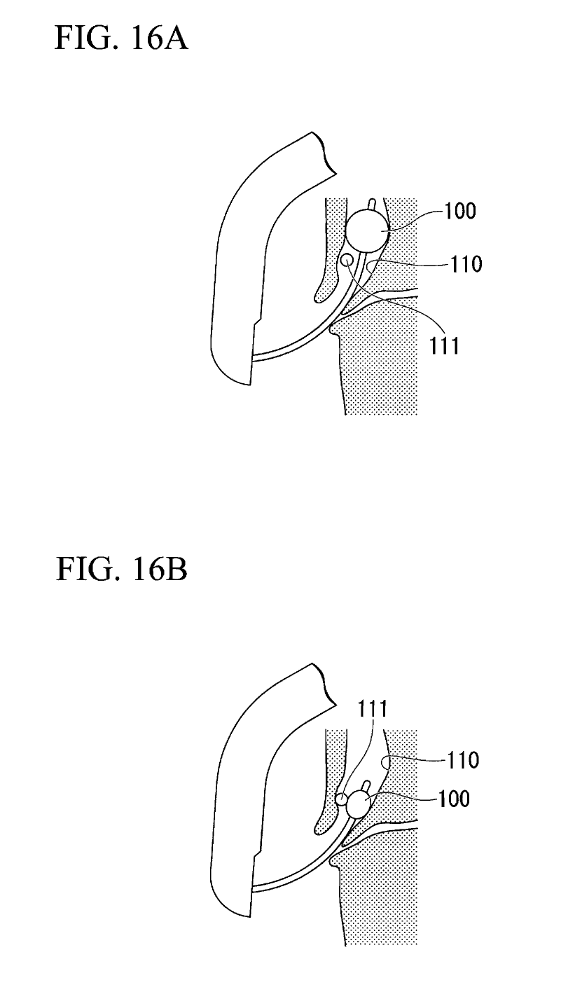

Next, a second embodiment of the present invention will be explained with reference to FIG. 13. A fluid feeder 31 according to this embodiment differs from the proceeding fluid feeder 1 with regard to the structure of the adjustor 14.

In the following description, components that are the same as the first embodiment shall be provided with the same numeric symbol and redundant description shall be omitted.

FIG. 13 is an enlarged view of an adjustor 32 and the engaged portion 19 of a fluid feeder 31. The size of each engaging projection 33 formed on the adjustor 32 differs and an engaging projection 33A provided on a proximal side of the adjustor 32 is larger than an engaging projection 33B provided on the distal side thereof. Accordingly, the size of the engaging projections 33 depends on its position on adjustor 32. The size increases as the position of the engaging projections 33 is closer to the proximal end of adjustor 32.

As the plunger 13 advances, the projection 19A of the engaged portion 19 engages with the engaging projections 33 positioned closer to the proximal side. As a result, more fluid is supplied into the balloon 4 so as to increase the diameter of the balloon 4. At this time, tension exerted on the balloon 4 increases so as to increase a force exerted toward the retracting direction of the plunger 13 in conjunction with the increase of the diameter of the balloon.

According to the fluid feeder 31 of the present embodiment, since the size of the engaging projections positioned closer to the proximal side of the adjustor 32 is designed to increase, an engagement force exerted between the projection 19A and the engaging projections 33 (a force required for the projection 19A rides over the engaging projections 33 abutted thereon) increases as a moving distance of the plunger 13 increases.

Therefore, although a stronger force exerts to the direction in which the plunger 13 is retracted with respect to the cylinder 12 by increasing a diameter of the balloon 4, the positional relationship between the plunger 13 and the cylinder 12 is maintained so as to maintain the diameter of balloon.

While preferred embodiments of the invention have been described and illustrated above, it should be understood that these are exemplary of the invention and are not to be considered as limiting. Additions, omissions, substitutions, and other modifications can be made without departing from the spirit or scope of the present invention.

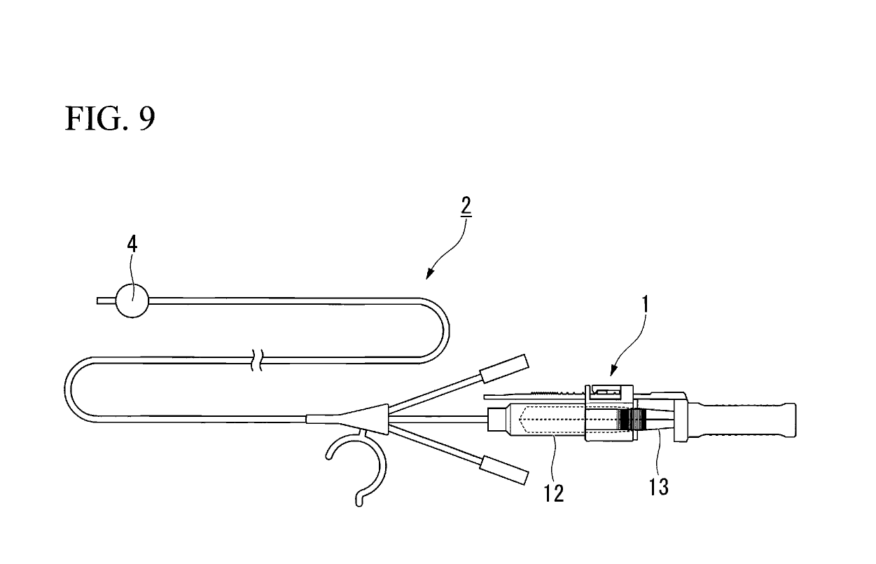

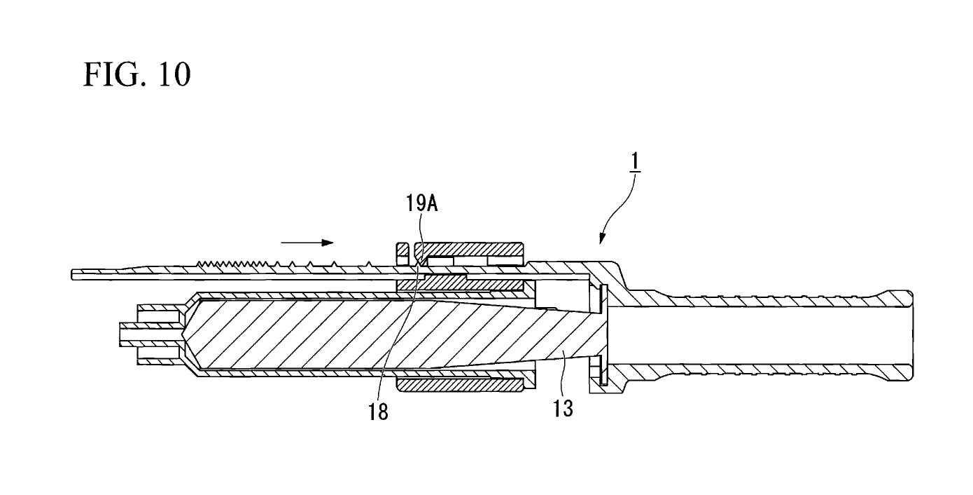

For example, as shown in FIGS. 10 and 13, in the proceeding embodiment, the angle of slant faces of both distal and proximal sides of the engaging projections of the adjustor were the same. However, the shape of the engaging projections is not limited thereto. Modified examples are described herein below.

FIG. 14 is an enlarged view of an adjustor 34 as a modified example of the present invention. An angle formed by a first slant face 35A (at a distal side) between a base of the adjustor 34 parallel to an axis of the plunger 13 (in other words, it is an angle formed by the first slant face 35A between the axis of the plunger 13) is set smaller than that of a second slant face 3513 (at the proximal side). As a result of this design, lesser force is required for the projection 19A to ride over the engaging projections 35 when the plunger 13 is advanced. Therefore, the inflation operation of the balloon 4 can be easily performed as well as having an advantage of reliably controlling the diameter of the balloon.

Alternatively, in the proceeding embodiment, the projection of the engaged portion and the engaging projections of an adjustor were engaged. However, in place thereof, it is also acceptable to design a concave portion 37 on an adjustor 36 which engages with the projection 19A of the engaged portion 19 as shown in a modified example of FIG. 15. In this case, in order to increase the engagement force at the concave portion 37 as the moving distance of the plunger 13 increases, a depth of the concave portion 37 may be increased as the position is closer to the proximal end of the adjustor 36.

Further, a fixing portion may be constructed by providing the concave portion at the engaged portion so as to engage with the engaging projections of the adjustor; or different angles may be formed at the first slant face and the second slant face between the axis of the plunger, as shown in the aforementioned modified example.

For example, the proceeding embodiments described examples in which a fluid feeder was used in the balloon catheter. However, the invention is not limited thereto; for example, the fluid feeder of the present invention may also be used in, for example, an inner diameter measuring device which measures an inner diameter of the tube by inflating a balloon in the tube so as to fit into. The inner diameter can be measured easily since the diameter of the balloon can be regulated at a desirable size.

Finally, the invention is not to be considered as being limited by the foregoing description and is only limited by the scope of the appended claims.

What is claimed is:

1. A fluid feeder which feeds a fluid into a balloon made of an elastic material so as to inflate, the fluid feeder provided with:
a cylinder that is formed in a cylindrical shape provided with a first port and a second port on each end of the cylinder and contains the fluid,
a plunger that is inserted into the cylinder via the second port in a freely advancing and retracting manner in an axial direction, in order to push out the fluid contained in the cylinder from the first port to the outside of the cylinder,
an adjustor that is attached to the plunger and regulates a moving distance of the plunger so as to inflate the balloon to a predetermined diameter, and
a fixing portion that is provided on the cylinder and fixes the plunger onto the cylinder to a position which corresponds to the moving distance regulated by the adjustor, wherein:
the adjustor has a plurality of engaging members which regulates the moving distance corresponding to a plurality of the different inflated diameters, and the fixing portion has an engaged portion which engages with the engaging members to fix the plunger.

2. A fluid feeder according to claim 1, wherein at least a portion of the plurality of the engaging member is constructed so as to increase a space between the adjacent engaging member as the inflated diameter of the balloon increases.

3. A fluid feeder according to claim 2, wherein at least a portion of the plurality of the engaging member is constructed so as to change an inflated diameter of the balloon by a predetermined amount.

4. A fluid feeder according to claim 1, wherein at least a portion of the plurality of the engaging member is constructed so as to increase an engagement force exerted between the engaging member and the engaged portion as the inflated diameter of the balloon increases.

5. A fluid feeder according to claim 1, wherein the engaging member has a first slant face (at a distal side) which is inserted into the cylinder and a second slant face (at the proximal side) along an axial direction of the plunger, an angle formed by the first slant face between the axis of the plunger is smaller than an angle formed by the second slant face between the axis of the plunger.

6. A balloon catheter made of an elastic material provides with the fluid feeder according to claims 1 or 2.
